(19) United States
(12) Patent Application Publication
(10) Pub. No.: US 2024/0102832 A1
(43) Pub. Date: Mar. 28, 2024

(54) METHOD FOR DETERMINATION OF A CONDITION OF AN OPTICAL FIBER OF AN ELECTROSURGICAL INSTRUMENT AND SYSTEM HAVING AN ELECTROSURGICAL INSTRUMENT

(71) Applicant: Erbe Elektromedizin GmbH, Tuebingen (DE)

(72) Inventors: Markus REITERER, Loipersbach (AT); Nikolaus DRAGOSTINOFF, Wien (AT); Alexander UNGER, Enzerdorf/Fischa (AT)

(21) Appl. No.: 18/470,636

(22) Filed: Sep. 20, 2023

(30) Foreign Application Priority Data
Sep. 22, 2022 (EP) 22197150.0

(51) Int. Cl. G01D 5/353; A61B 18/14

(52) U.S. Cl. CPC G01D 5/353; A61B 18/14; A61B 2018/00589

(57) ABSTRACT

A method and a system for determining a condition of an optical fiber cable of an electrosurgical instrument with regard to its transfer characteristic for light are described. The system can comprise an evaluation unit having a light source and a light analysis unit that can be optically coupled with the optical fiber cable. The emission light emitted from the light source is coupled in the optical fiber cable, reflected at its distal end and transmitted back to the light analysis unit and is received as receipt light. The receipt light is correlated with the emission light and therefrom a transfer characteristic can be determined. If this is carried out prior to the first use of the electrosurgical instrument and subsequently at least once, changes in the transfer characteristic can be determined and the condition of the optical fiber cable can be concluded.

### CROSS REFERENCE TO RELATED APPLICATION

This application claims the benefit of European Patent Application No. 22197150.0, filed Sep. 22, 2022, the contents of which are incorporated herein by reference as if fully rewritten herein.

### TECHNICAL FIELD

The invention refers to a method for determination of a condition of an optical fiber cable of an electrosurgical instrument. The invention also refers to a system comprising an electrosurgical instrument.

### BACKGROUND

By means of electrosurgical instruments human or animal tissue can be influenced by means of an electrical discharge. For this purpose the electrosurgical instrument comprises at least one electrode. For example, it is possible to coagulate or cut by means of the electrosurgical instrument.

EP 2 815 695 A1 describes an electrosurgical instrument having an electrode. During treatment of tissue light is created at the electrode that is transmitted by means of an optical fiber to an evaluation unit for spectral analysis. In the evaluation unit the tissue type of the treated tissue can be determined based on the light.

A method for determination of the tissue type is also known from EP 3 964 822 A1. There light transmitted via the optical fiber from the electrosurgical instrument to an evaluation unit is evaluated and a transmission degree is determined. In doing so, it is possible to determine the contamination of the electrosurgical instrument at the light entrance window and thereby to determine the reliability for determination of the tissue type.

A method and a device for diagnosis and monitoring of the transmission quality of a fiber optical system is described in DE 198 40 346 A1. A laser treatment instrument is connected with a laser light source via an optical fiber that supplies the laser instrument with laser light. The optical fiber is monitored continuously in order to recognize damages. By means of an additional test light source, test light can be coupled into the optical fiber and reflected light can be received and evaluated in order to determine the transmission quality. The received light is thereby guided over a polarization beam splitter in order to remove components of reflected light that are created prior to coupling the test light into the optical fiber. A similar method is also known from EP 3 949 887 A1. There the laser treatment instrument comprises a reflection unit at the distal end of the optical fiber for reflecting light that has been coupled therein.

In the method for evaluation of the integrity of an optical fiber described in EP 2 823 278 A2 a reflection at a fiber end is separated from the determined total reflection of the light coupled into the optical fiber in order to obtain a calibrated intensity measurement for the integrity evaluation of the optical fiber.

U.S. Pat. No. 5,965,877 A proposes to evaluate the integrity of an optical fiber by use of light having different wavelengths. During the use of operation light, test light can be coupled into optical fibers that, at the opposite fiber end, excites luminescent material to luminate, wherein this light comprises an additional different wavelength. The reflection light created by luminescence is transmitted back through the optical fiber and can be evaluated in order to draw a conclusion about the integrity of the optical fiber.

CN 209264547 U describes a fiber coupler having three connections. One connection is connected with a light source, another connection is connected with a detector and a third connection is connected with a gas measurement cell. Light can be transmitted from the light source to the gas measurement cell, can be reflected there and can be transmitted back to the detector. For the light path to the gas measurement cell and for the light path from the gas measurement cell back to the detector different fibers of the fiber optical cable are used.

Additional methods and devices for evaluation of a condition of an optical fiber cable are described in U.S. Pat. No. 4,716,288 A or U.S. Pat. No. 5,219,345 A.

In addition, U.S. Pat. No. 7,132,645 B2 and EP 0 926 479 A1 describe systems and methods for determining losses in a light path comprising an optical fiber.

### SUMMARY

Starting from the prior art it can be considered as one object of the present invention to provide a method for determination of a condition of an optical fiber cable of an electrosurgical instrument that can be realized economically using simple means. In addition, it is an object of the invention to provide a system by means of which the method can be carried out in a simple and economic manner.

These objects are solved by means of the methods and systems described herein.

The method according to one aspect of the invention is configured to determine the condition of an optical fiber of an electrosurgical instrument. The electrosurgical instrument comprises at least one electrode. It can be configured as monopolar or bipolar electrosurgical instrument.

For supply with electrical energy of the at least one electrode, the electrosurgical instrument is electrically connected with an energy source of a supply apparatus. For this purpose the electrosurgical instrument can comprise at least one electrical conductor that connects the electrode with the energy source respectively.

The electrosurgical instrument comprises in addition an optical fiber cable having a distal end as well as a proximal end. For example, the distal end is attached to a handle piece of an electrosurgical instrument and can comprise a distal face that serves as light entrance window for capturing light at the at least one electrode. Via the optical fiber cable the electrosurgical instrument can be optically connected with an evaluation unit. The evaluation unit can be part of the supply apparatus.

For evaluation of the condition of the optical fiber cable, particularly the contamination of the light entrance window or the distal end of the optical fiber cable, the electrosurgical instrument is first provided in an initial condition. In the initial condition the electrosurgical instrument can be new or can be cleaned or sterilized in the case of reusable instruments. The electrosurgical instrument can be configured as single-use instrument or also as reusable instrument that can be reused.

In the initial condition the electrosurgical instrument is electrically connected with the energy source and optically connected with the evaluation unit. Subsequently, a transfer characteristic is determined that serves as reference characteristic in an electrosurgical instrument being in a not damaged and not contaminated initial condition.

Subsequently the electrosurgical instrument can be used for treatment of animal or human tissue of a patient. For this purpose the electrosurgical instrument is activated, wherein the at least one electrode is supplied with electrical energy from the energy source. At the at least one electrode an electrical discharge is created, e.g. a spark or a light arc, by means of which tissue can be influenced, e.g. in order to coagulate or cut the tissue.

After deactivation of the electrosurgical instrument
whereby the supply of the at least one electrode with electrical energy is cut off—again a transfer characteristic is determined as current condition characteristic. This determination of a current condition characteristic can be carried out whenever a predetermined criteria is fulfilled. The predetermined criteria can be fulfilled in one of the following cases, for example:
   after having the electrosurgical instrument activated and deactivated one single time respectively;
   after a predetermined number of activations and deactivations of the electrosurgical instrument respectively, wherein the number is at least two;
   after a predetermined total activation duration of the electrosurgical instrument has been achieved.

Subsequently by means of comparison of the current condition characteristic with the initially determined reference characteristic the condition and particularly the condition of contamination of the distal end of the optical fiber cable can be determined.

For this determination of the present condition of the optical fiber cable no complex devices are required. The electrosurgical instrument provided for treatment is checked in the initial condition and in addition after use with regard to its transfer characteristic, whereby the change of condition compared to the initial condition can be determined. For example, if due to wear, contamination, damage or the like a deviation of the condition of the optical fiber cable compared to the initial condition is determined, measures can be initiated. For example, a cleaning or a replacement of the electrosurgical instrument can be arranged. For this purpose the output of a respective notification to an operating person can be caused by means of the evaluation unit.

For determination of a transfer characteristic as reference characteristic or as current condition characteristic it is proceeded as follows:

A light source of the evaluation unit emits light that can be denoted as emission light. The emission light is at least partly coupled into the proximal end of the optical fiber cable and is there guided in direction to the distal end. At least a part of the emission light reaching the distal end, is reflected there and guided back through the optical fiber cable toward the proximal end. At the proximal end at least part of the reflected light is decoupled and transmitted to the evaluation unit. The light reaching the evaluation unit can be denoted as receipt light. By means of an evaluation based on the emission light and the receipt light, the transfer characteristic can be determined. For example, a reflectance can be determined in that the intensity of the receipt light is compared with the intensity of the emission light. The transfer characteristic can depend on the wavelength. For example, it can describe a reflection spectrum or a reflectance for multiple wavelengths.

The method is preferably configured to receive light that is created by sparks or light arc formation at the at least one electrode—for example during an electrosurgical intervention on human or animal tissue—at least partly at the distal end of the optical fiber cable and to transfer it to the evaluation unit. In the evaluation unit the receipt light can be analyzed, particularly by means of spectral analysis using a light analysis unit of the evaluation unit, e.g. a spectrometer. Based on the evaluation of the receipt light, at least one tissue parameter can be determined, e.g. a tissue type. Particularly the spectral composition of the light is different during treatment of differential tissue types, wherein tissue types, such as fat tissue, muscle tissue, bone tissue, etc. can be distinguished from one another. It is thereby possible to indicate the presently treated tissue type respectively to the operating person or surgeon.

It is advantageous, if the light source is activated to emit emission light only, if no electrical energy applies to the at least one electrode. In doing so, it can be guaranteed that during capture of light by an electrical discharge at the electrode and the transmission of light from the distal end of the optical fiber cable to the evaluation unit no influence of emission light of the light source occurs. For example, a tissue analysis during treatment of tissue is then improved.

It is advantageous, if the emission light of the light source has light wavelengths in the range of below 380 nm. The emission light of the light source can have multiple maxima at different light wavelengths, wherein the maxima can have different intensities. For example, a principle maximum can occur at light wavelengths of more than 400 nm, e.g. in the range between 450 nm and 460 nm. The emission light can have a secondary maximum as an option in the range of 340 nm to 360 nm.

An inexpensive light-emitting diode can be used as light source, e.g. a light-emitting diode of the type MLEROY-A1-0000-000501.

Preferably the evaluation unit comprises a light analysis unit for analysis of light that can be particularly configured as spectrometer and preferably as slit spectrometer. For optical coupling of the evaluation unit with an optical fiber of the supply apparatus or the optical fiber of the electrosurgical instrument a coupling unit can be present. The coupling unit can be configured as 2:1-coupler and can thus have three connections: a first connection for the light source, a second connection for the light analysis unit and a third connection for the optical fiber of the supply apparatus or the electrosurgical instrument.

In a first embodiment an optical fiber cable of the supply apparatus is at an end connected to the third connection of the coupling unit and is connected at an opposite other end with an apparatus connection of the supply apparatus. At the apparatus connection (e.g. socket) the optical fiber cable of the instrument can be connected by means of a connection element (e.g. plug).

In variation thereto the coupling unit can be part of the apparatus connection of the supply apparatus in a second embodiment, so that the optical fiber cable of the instrument can be connected to the third connection of the coupling unit by means of the connection element.

In all embodiments a contamination at the distal end of the optical fiber cable of the instrument or another impairment of the optical fiber cable of the instrument can be determined. In addition, in the first embodiment also a contamination or another impairment at the apparatus connection can be determined.

When using such a coupling unit, the first connection and the second connection can be optically separated from one another so that a direct optical transmission from the first connection to the second connection by means of the coupling unit only, can be excluded. A transmission of light from the first connection to the second connection or vice versa is particularly only possible, if one of the optical fibers is optically connected with the third connection.

When using such a coupling unit, emission light can be coupled into the first connection of the coupling unit. In the evaluation unit it can be checked whether receipt light is received and/or a feature of the receipt light (e.g. intensity or luminosity) can be evaluated. If receipt light is received, it can be determined in the evaluation unit—at least when using the second embodiment—that an electrosurgical instrument is connected with its optical fiber cable to the third connection of the coupling unit (as part of the apparatus connection). In the first embodiment it can be checked by means of the evaluation of the feature of the receipt light whether the electrosurgical instrument is connected to the apparatus connection. For example, the intensity or luminosity of the receipt light can be compared with the intensity or luminosity of the emission light. The intensity or luminosity of the receipt light increases, if in addition at the transition from the optical fiber cable of the supply apparatus to the optical fiber cable of the instrument and/or at the distal end of the optical fiber cable of the instrument a portion of the emission light is reflected respectively.

By repeated emission of emission light and coupling the emission light into the first connection, it can be thus checked and determined whether the electrosurgical instrument is connected to the coupling unit so that subsequently the reference characteristic can be determined.

Alternatively to this, the determination of the reference characteristic can also be triggered by the operating person by means of an operating interface, e.g. in the case where an automatic recognition of an instrument connected to the supply apparatus is not implemented.

A system according to the invention can be preferably configured for carrying out of any embodiment of the above-described method. The system comprises a supply apparatus having an energy source for supply of the at least one electrode of the electrosurgical instrument. In addition, the system comprises an evaluation unit. The evaluation unit can be part of the supply apparatus or can also be realized separately from the supply apparatus. The evaluation unit in turn comprises a light source and a light analysis unit (e.g. spectrometer) for analysis of light. As explained, an inexpensive slit spectrometer can be preferably used as spectrometer. By means of the slit of the slit spectrometer, the receipt light can be separated in its spectral components.

The electrosurgical instrument comprises an optical fiber cable that is optically coupled by means of a coupling unit with the evaluation unit and thus with the light source as well as with the light analysis unit. Thereby the light source is optically connected to the first connection of the coupling unit, the light analysis unit is optically connected to the second connection of the coupling unit and the optical fiber cable of the instrument is indirectly (e.g. via an optical fiber cable of the supply apparatus) or directly optically connected to a third connection of the coupling unit.

Here it has to be pointed out that the numbering of the connections serves for distinction only and does not impose a limitation with regard to the number of connections or the sequence or priority of the connections.

The coupling unit is configured so that no direct optical transmission between the first connection and the second connection occurs. The first connection and the second connection are optically separated and are optically individually connected with the third connection of the coupling unit respectively.

The configuration of this coupling unit and the components connected thereto, particularly the features described in the following in this regard, is a separate aspect of the invention that can also be realized independently from the configuration of the method and the system according to the invention.

It is preferred, if the coupling unit comprises a fiber bundle having at least one first optical fiber and at least one second optical fiber. The at least one first optical fiber optically connects the first connection of the coupling unit with the third connection of the coupling unit. The at least one second optical fiber optically connects the second connection of the coupling unit with the third connection of the coupling unit. Preferably exactly one first optical fiber can be present. Further preferably multiple second optical fibers are present. At the third connection and in an area of the coupling unit adjoining the third connection, the first optical fiber is preferably arranged centrally and the multiple second optical fibers can be distributed in circumferential direction around the first optical fiber and can be particularly uniformly distributed. It is advantageous, if at least three second optical fibers are present. In a preferred embodiment four second optical fibers are present.

It is advantageous, if the optical axis of the first optical fiber is orientated along the optical axis of the optical fiber cable, if the optical fiber cable is connected to the third connection of the coupling unit.

It is advantageous, if the core cross-section area of the at least one first optical fiber and the at least one second optical fiber are respectively smaller than the core cross-section area of the optical fiber cable connected to the third connection. The core cross-section area of the optical fiber cable connected to the third connection has a circumferential contour inside which all of the core cross-section areas of the at least one first optical fiber and the at least one second optical fiber are arranged.

Advantageously, the at least one first optical fiber has a core cross-section area that is smaller than the core cross-section area of the at least one second optical fiber. For example, the core cross-section area of the at least one first optical fiber can be maximum 25% of the core cross-section area of the at least one second optical fiber. Additionally or alternatively, the core cross-section area of the at least one second optical fiber can be maximum 25% of the core cross-section area of the optical fiber cable.

The evaluation unit can comprise an arrangement for mode mixing. The arrangement for mode mixing is arranged between the evaluation unit and the apparatus connection in the first embodiment and the coupling unit in the second embodiment. The light transmitted between the evaluation unit and the coupling unit is thus mixed with regard to the present modes. This applies to the emission light transmitted to the coupling unit as well as to the receipt light transmitted from the coupling unit to the evaluation unit.

### BRIEF DESCRIPTION OF THE DRAWINGS

Advantageous embodiments of the invention are derived from the dependent claims, the description and the drawing. In the following, preferred embodiments of the invention are explained in detail based on the attached drawing. The drawing shows:

FIG. 1 a block diagram of a first embodiment of the system according to the invention comprising an energy source, an evaluation unit and an electrosurgical instrument,

FIG. 2 a block diagram-like principle illustration of the electrosurgical instrument that is connected via a coupling unit to the evaluation unit according to the first embodiment according to FIG. 1,

FIG. 3 a block diagram of a second embodiment of the system according to the invention comprising an energy source, an evaluation unit and an electrosurgical instrument,

FIG. 4 a block diagram-like principle illustration of the electrosurgical instrument connected to the evaluation unit via a coupling unit according to the second embodiment according to FIG. 3,

FIG. 5 a basic illustration of the arrangement of at least one first fiber and at least one second fiber of the coupling unit of FIG. 2 at a third connection of the coupling unit,

FIG. 6 a basic illustration of a proximal face of an optical fiber cable of the electrosurgical instrument,

FIG. 7 a basic illustration of the arrangement of multiple second optical fibers of the coupling unit according to FIGS. 2 and 3 at a second connection of the coupling unit of FIG. 2,

FIG. 8 a general principle illustration of a preferred arrangement possibility of optical fibers at an arbitrary coupling location,

FIG. 9 an exemplary spectrum of emission light of light source of the evaluation unit,

FIG. 10 an exemplary transfer characteristic of an optical fiber cable or an electrosurgical instrument depending on a light wavelength and

FIG. 11 a flow diagram of an embodiment of a method according to the invention.

### DETAILED DESCRIPTION

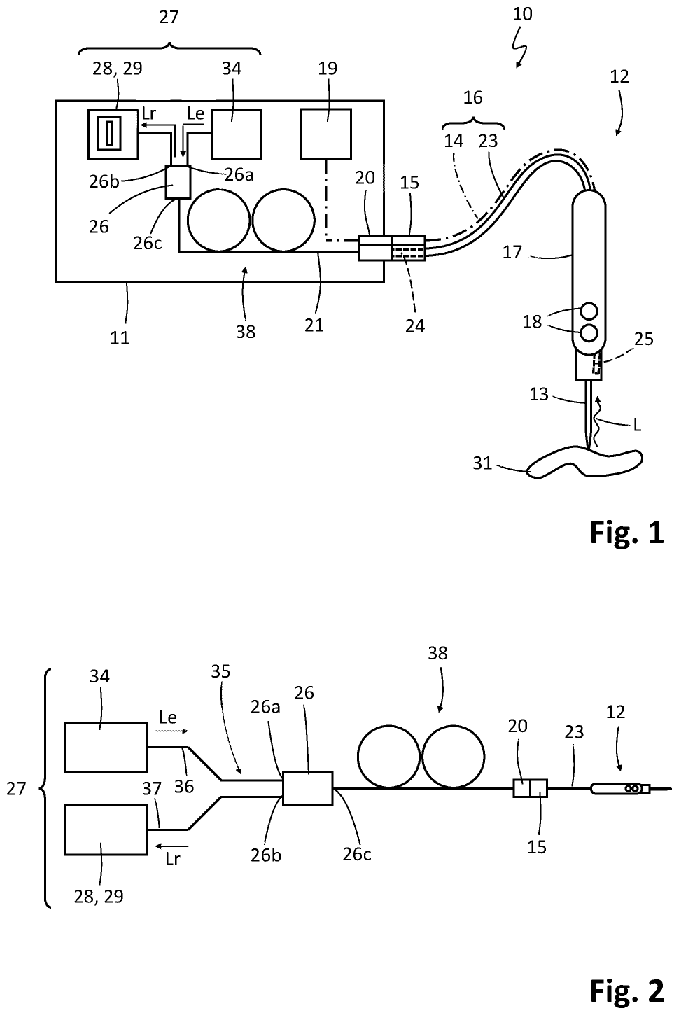

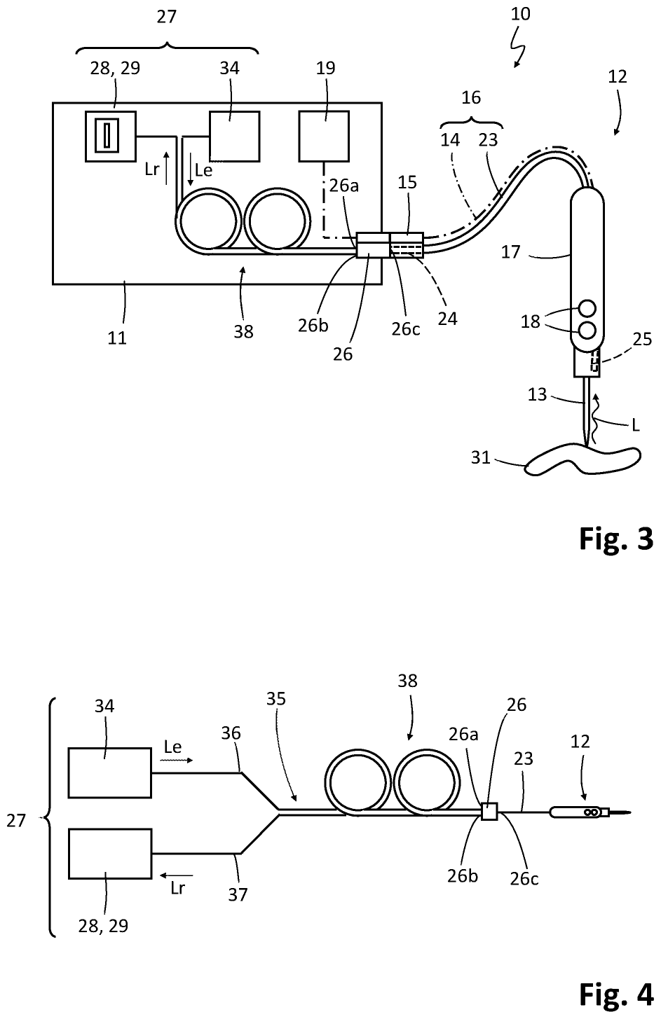

In FIGS. 1 and 2 a first embodiment of a system 10 is schematically illustrated and in FIGS. 3 and 4 a second embodiment of system 10 is illustrated. The system 10 comprises a supply apparatus 11 and an electrosurgical instrument 12 connected or connectable thereto.

The electrosurgical instrument 12 can be configured as a monopolar or bipolar instrument and comprises at least one electrode 13. The at least one electrode 13 is electrically connected via an assigned electrical conductor 14 to a connection element 15. The electrical conductor 14 can be part of a cable 16. The cable 16 extends between the connection element 15 and a handle piece 17 of the electrosurgical instrument 12.

The at least one electrode 13 is attached to the handle piece 17. In the embodiment one or multiple operating elements 18 are provided at the handle piece 17 by means of which the electrosurgical instrument 12 can be activated and deactivated. In addition to the at least one operating element 18 on the handle piece 17 also a separate foot switch or the like can be provided in order to activate or deactivate the electrosurgical instrument 12.

By means of the supply apparatus 11, the at least one electrode 13 is supplied with electrical energy in the activated condition. For this purpose the supply apparatus 11 comprises an energy source 19. The energy source 19 is electrically connected to an apparatus connection 20. The apparatus connection 20 is configured to be connected with the connection element 15. In doing so, an electrical connection between the energy source 19 and the at least one electrode 13 can be established via the at least one electrical conductor 14.

The electrosurgical instrument 12 comprises in addition an optical fiber cable 23. The optical fiber cable 23 of the electrosurgical instrument 12 has a proximal end 24 connected to the connection element 15. A distal end 25 of optical fiber cable 23 is attached to handle piece 17, preferably by means of a substance bond connection, e.g. an adhesive connection. The distal end 25 could be additionally or alternatively attached to the handle piece 17, also in a force-fit and/or form-fit manner. A face at the distal end 25 of optical fiber cable 23 serves as light-receiving window and faces electrode 13. Light entering the distal end 25 can be guided via the optical fiber cable 23 to the proximal end 24.

The proximal end 24 of optical fiber cable 23 of the electrosurgical instrument 12 can be optically coupled with a coupling unit 26 via connection element 15 and apparatus connection 20 and via the coupling unit 26 with an evaluation unit 27. The coupling unit 26 can be arranged in the interior of the housing of supply apparatus 11 and can be optically connected to the apparatus connection 20 via an optical fiber cable 21 of supply apparatus 11 (first embodiment according to FIGS. 1 and 2) or can be part of the apparatus connection 20 (second embodiment according to FIGS. 3 and 4). By connecting the connection element 15 with apparatus connection 20, in both cases an optical coupling can be established between proximal end 24 and the coupling unit 26 and thereby also with the evaluation unit 27.

Alternatively to the illustrated embodiment, it is also possible to realize the electrical connection and the optical connection between electrosurgical instrument 12 and supply apparatus 11 by means of multiple separate connection elements 15 and separate apparatus connections 20.

In the embodiment the evaluation unit 27 is part of supply apparatus 11. In a modified embodiment the evaluation unit 27 could also be arranged separate from supply apparatus 11 in an individual housing.

The evaluation unit 27 comprises a light analysis unit 28, which is configured as spectrometer 29 and particularly as slit spectrometer in the embodiment, for evaluation of light that has been transmitted from the electrosurgical instrument 12. By means of a slit 30 of spectrometer 29 (FIG. 7), the receipt light can be divided into its contained light wavelengths A and the intensities of the contained light wavelengths A can be evaluated individually.

During use of the electrosurgical instrument 12 during treatment of human or animal tissue 31, an electrical discharge occurs at the at least one electrode 13, e.g. a spark and/or a light arc. The light L created thereby can partly enter into the face at distal end 25 of optical fiber cable 23 and is further transmitted via proximal end 24 at connection element 15 via coupling unit 26 to the light analysis unit 28 of evaluation unit 27. There at least one tissue parameter of the treated tissue 31 can be determined, e.g. the tissue type, by means of analysis of the light, particularly the spectral analysis of the light. Thereby different tissue types, such as muscle tissue, fat tissue, bone tissue, etc. can be distinguished from one another and can be indicated to the operating person or the surgeon by means of a suitable interface.

In addition a light source 34 is part of the evaluation unit 27 in the embodiment. The light source 34 and the light analysis unit 28 are optically connected with coupling unit 26 by means of a fiber bundle 35. The fiber bundle 35 comprises at least one first optical fiber 36 and at least one second optical fiber 37. The at least one first optical fiber 36 is at one end optically connected to the light source 34 and at another end optically connected to a first connection 26a of coupling unit 26. The at least one second optical fiber 37 is at one end optically connected to the light analysis unit 28 and at another end optically connected to a second connection 26b of coupling unit 26. The optical fiber cable 21 of supply apparatus 11 (FIGS. 1 and 2) or the optical fiber cable 23 of instrument 12 (FIGS. 3 and 4) is optically connected to a third connection 26c of coupling unit 26.

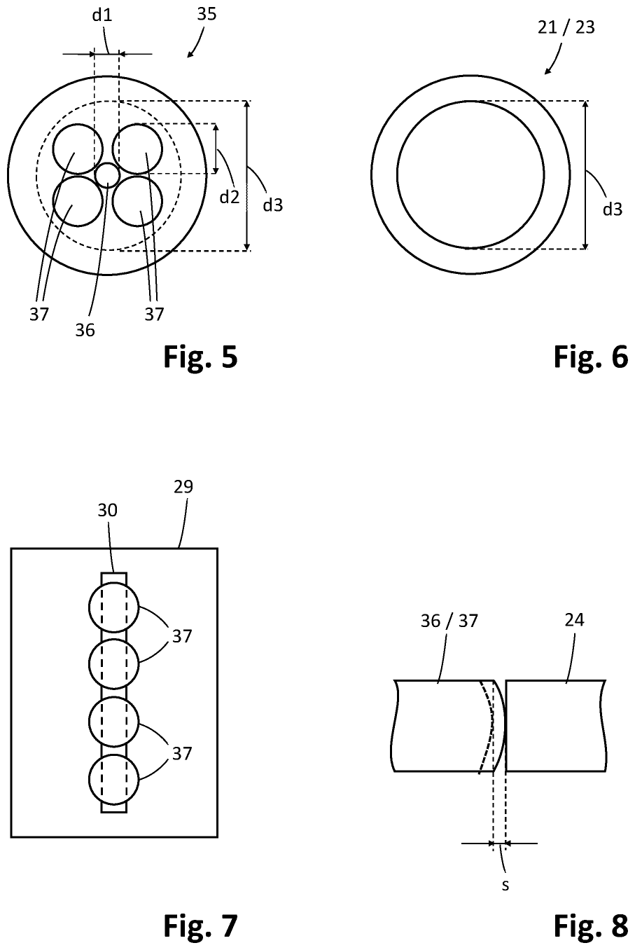

According to the example, fiber bundle 35 comprises exactly one first optical fiber 36 and multiple second optical fibers 37, preferably at least three first optical fibers 37 and in the embodiment four second optical fibers 37. Within fiber bundle 35 the first optical fiber 36 extends along a longitudinal center axis of fiber bundle 35, as schematically illustrated in FIG. 5. The multiple second optical fibers 37 are distributed in circumferential direction around the first optical fiber 36. For optical connection with evaluation unit 27 or coupling unit 26 respectively, the optical fiber bundle 35 is separated in the area of its respective end.

As apparent from FIG. 5, the core cross-section area and according to the example the core diameter of the at least one first optical fiber 36 is smaller than the core cross-section area or the core diameter of the at least one second optical fiber 37. The core cross-section area or core diameter of the at least one first optical fiber 36 and the at least one second optical fiber 37 are in turn smaller than the core cross-section area or the core diameter of the optical fiber cable 21, 23 connected to the third connection 26c (FIG. 6).

In the embodiment for better distinction core diameter of the at least one first optical fiber 36 is denoted as first diameter d1, core diameter of the at least one second optical fiber is denoted as second diameter d2 and core diameter of the optical fiber cable 21 or 23 is denoted as third diameter d3.

According to the example, third diameter d3 is at least as large or larger than the sum of the first diameter d1 and two times second diameter d2. In doing so, it is possible to arrange the one first optical fiber 36 and the four second optical fibers 37 in a manner, so that they are located entirely inside a circular area defined by a third diameter d3 (FIG. 5).

For example, third diameter d3 can be 600 μm, second diameter d2 can be 200 μm and first diameter d1 can be 100 μm.

The optical fiber cable 21, 23 is configured as multi-mode fiber (MMF) according to the example. In addition, or as an alternative, also the at least one optical fiber 36 and/or the at least one second optical fiber 37 can be configured as multi-mode fiber respectively. In this case it is advantageous, if an arrangement 38 for mode mixing is provided between the evaluation unit 27 and the coupling unit 26 (FIGS. 1-4). For example, the arrangement 38 for mode mixing can be created by arranging of optical fiber cable 21 of supply apparatus 11 (FIGS. 1 and 2) or fiber bundle 35 (FIGS. 3 and 4) in at least one arc and/or at least one loop. Preferably multiple arcs or loops are arranged in spatially different planes, e.g. in three planes arranged orthogonal to one another. The arcs can be arranged circularly, elliptically or in the shape of an “8” or the like.

As schematically depicted in FIG. 7, the second optical fibers 37 are arranged along a line next to one another in extension direction of slit 30. In this manner a large portion of the slit area can be covered by the multiple second optical fibers and a large amount of light can be coupled into spectrometer 29 through slit 30. In doing so, the spectral analysis is improved and is still possible with low amounts of light that are received at the distal end 25 of optical fiber cable 23 during operation of the electrosurgical instrument 12.

In FIG. 8 a preferred manner of an optical coupling between two fiber ends is schematically illustrated. The optical coupling can be used at each position of system 10, where two fiber ends shall be optically coupled with each other. The fiber ends are preferably in contact or are arranged at a distance s opposite one another (shown in dashed lines). The optional distance s represents the minimum distance between the faces of the fiber ends. The optical axes of the two fiber ends are preferably parallel or congruent. The face of one fiber end is a planar surface, while the face of the other fiber end is a convex surface. The convex face can be a polished surface. The planar surface of the respective other fiber end can be a polished planar surface or a not post-treated fracture surface. The fiber end having the planar face can be one of the optical fiber cables 21, 23, for example, while the other fiber end having the convex face can be the fiber end of first fiber 36 and/or a second fiber 37. Vice versa it is also possible to configure the fiber ends of the at least one first optical fiber 36 and/or the at least one second optical fiber 37 in a planar manner and to configure the face at the proximal end 25 of an optical fiber cable 21, 23 in a convex manner.

The light source 34 emits light that can be denoted as emission light Le. For example, a light emitting diode can be used as light source 34, e.g. a UV-light-emitting diode that emits emission light Le, at least also in the UV-wavelength range.

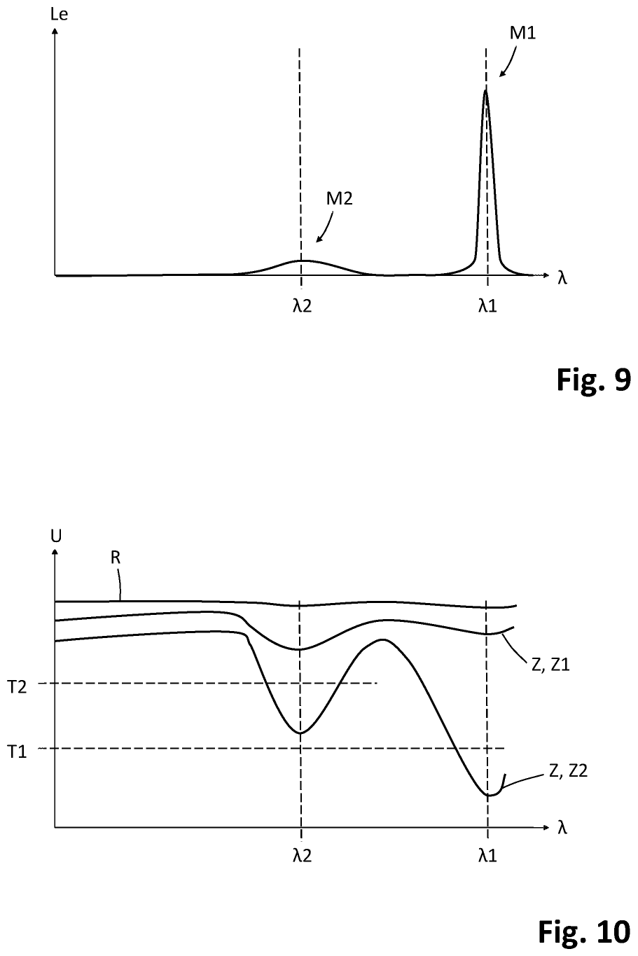

An exemplary spectrum of emission light Le is illustrated in FIG. 9. The emission light Le comprises preferably light wavelengths λ in a range of less than 380 nm. In the embodiment the spectrum of the emission light Le has a first maximum M1 at a first light wavelength λ1 and a second maximum M2 at a second light wavelength λ2. The first light wavelength λ1 is in the range of 400 nm to 500 nm and in the embodiment in the range of 450 nm to 460 nm. The second light wavelength λ2 is smaller than 380 nm and is, according to the example, in the range between 340 nm and 360 nm, for example approximately 350 nm. Such a spectrum can be obtained with use of an inexpensive light-emitting diode. The first maximum M1 is, according to the example, the principle maximum and the second maximum M2 the secondary maximum of the spectrum of the emission light Le.

In addition or as an alternative to the second maximum M2, the emission light Le could also contain light having a wavelength in the range between 200 nm and 260 nm, which can provide additional advantages for the evaluation of the transfer characteristic U of the electrosurgical instrument 12 or the optical fiber cable 23, because here differences in the transmission of light depending on a degree of contamination of the distal end 25 become particularly clear.

The spectrum of emission light Le illustrated in FIG. 9 and used according to the example is only exemplary. The spectrum of the emission light Le could also have another spectral composition.

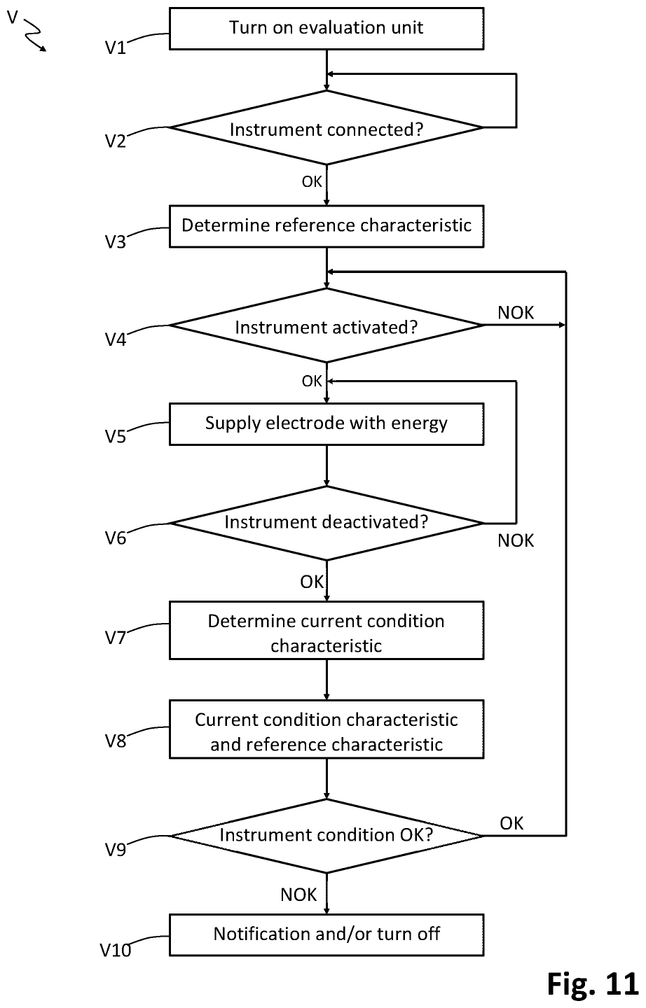

An embodiment of a method V is illustrated in FIG. 11. The method V is configured to determine the condition of the optical fiber cable 23 based on its transfer characteristic U, particularly a contamination of the distal end 25 and/or other damages or impairments that influence the light transmission. With increasing contamination or when other damages or wear of optical fiber cable 23 occurs, the spectrum of the transmitted light can be influenced so that the determination of a tissue parameter by means of the light analysis unit 28 is no longer possible with sufficient reliability, for example. By means of the method V, such an impairment can be recognized and a suitable measure can be initiated, e.g. a cleaning of the electrosurgical instrument 12 or the distal end 25 of optical fiber cable 23 or also an exchange of the electrosurgical instrument 12.

In a first method step V1 the evaluation unit 27 is turned on. In an optional second method step V2 it is checked whether a new or cleaned or sterilized electrosurgical instrument 12—i.e. an electrosurgical instrument 12 in an initial condition—is connected to the evaluation unit 27. Only if the electrosurgical instrument 12 is connected (branch OK out of second method step V2). The method is continued in a third method step V3. The established connection of instrument 12 to the supply apparatus 11 can alternatively also be confirmed by an operating person by means of an appropriate operating interface, in order to continue the method in the third method step V3.

In the third method step V3 a transfer characteristic U for the light guided through the optical fiber cable 23 is determined for the first time. The transfer characteristic U can in addition depend on the optical coupling between connection element 15 and coupling unit 26 and/or the optionally provided arrangement 38 for mode mixing and/or at least one additional optical influence parameter. However, the transfer characteristic U is at least influenced also from the condition of the optical fiber cable 23 and particularly the contamination of the distal end 25 of optical fiber cable 23. In the first embodiment, in addition, also a contamination of apparatus connection 20 can be checked and determined, because thereby the light reflection at an end of the optical fiber cable 21 of the supply apparatus 11 assigned to the apparatus connection 20 is influenced. A contamination at the apparatus connection 20 can be distinguished from a correctly connected and optically coupled optical fiber cable 21 of instrument 12 in the light analysis unit 28. Other influence parameters are substantially invariant during operation of system 10, so that a change in the transfer characteristic U can be at least substantially associated with the condition of the optical fiber cable 23.

After connection of an electrosurgical instrument 12 in the initial condition (new or sterilized or cleaned) the transfer characteristic U determined in the third method step V3 is a reference characteristic R, as illustrated in FIG. 10 by way of example. If the electrosurgical instrument 12 is in the initial condition in which the optical fiber cable 23 is neither contaminated nor damaged, the light is transmitted through the optical fiber cable 23 substantially independent from the light wavelength A according to the example.

To determine the transfer characteristic U, it is proceeded as follows according to the example:

The light source 34 is activated and emission light Le is at least partly coupled into the proximal end 24 of optical fiber cable 23 by means of coupling unit 26 (and the optional present optical fiber cable 21 of supply apparatus as well as the apparatus connection 20). In the optical fiber cable 23 light is forwarded to the distal end 25 and is partly reflected there. The reflected light is guided back in direction toward proximal end 24 and is there transmitted to the light analysis unit 28 (e.g. spectrometer 29) by means of coupling unit 26. The light reaching the light analysis unit 28 can be denoted as receipt light Lr. The receipt light Lr and the emission light Le are then correlated with one another in order to determine the transfer characteristic U. In the embodiment the intensities of multiple light wavelengths λ are compared so that the transfer characteristic U is a reflectance of the light transmission.

After determination of reference characteristic R method V is continued in a fourth method step V4, in which it is checked whether the electrosurgical instrument 12 has been activated for treatment of tissue 31. The activation can be carried out by the operating person or the surgeon, e.g. by means of the at least one operating element 18 on the handle piece 17. In the activated condition of the electrosurgical instrument 12 (branch OK out of fourth method step V4) the at least one electrode 13 is supplied with electrical energy, particularly a high frequency voltage is applied (fifth method step V5). For example, tissue 31 can then be treated by means of an electrical discharge created at the at least one electrode 13, for example by coagulation or cutting.

In a sixth method step V6 it is checked whether the instrument has been deactivated again. As long as this is not the case (branch NOK out of the sixth method step V6), the at least one electrode 13 is continuously supplied with electrical energy (fifth method step V5). Only after deactivation of the electrosurgical instrument 12 (branch OK out of sixth method step V6), the method is continued in a seventh method step V7 and a transfer characteristic U is determined as condition characteristic Z again. Examples for a first condition characteristic Z1 and a second condition characteristic Z2 are schematically depicted in FIG. 10.

After determination of the current condition characteristic Z in the seventh method step V7, in an eighth method step V8 the current condition characteristic Z is compared or correlated with reference characteristic R determined in the third method step V3. If differences between the current condition characteristic Z and the reference characteristic R are sufficiently small, it is determined in a ninth method step V9 that the condition of the electrosurgical instrument allows continuation of operation (branch OK out of ninth method step V9 to fourth method step V4).

If however a deviation of the current condition characteristic Z from the reference characteristic R is determined that fulfills a predetermined criteria, a condition that does not allow continuation of operation (e.g. contamination) is determined (branch NOK out of ninth method step V9). In all embodiments such a contamination can be determined at the distal end 25 of optical fiber cable 21 of instrument 12 and in the first embodiment also at the apparatus connection 20. Then, in a tenth method step V10, a suitable measure can be requested or automatically initiated, e.g. a notification to an operating person or the surgeon and/or switching off the energy supply of the electrosurgical instrument 12. For example, then an exchange of the electrosurgical instrument 12 or a cleaning can be carried out in order to resume operation.

In the case of first condition characteristic Z1 shown by way of example in FIG. 10, the differences compared with reference characteristic R are sufficiently small, so that it is determined in the ninth method step V9 that the condition of the electrosurgical instrument according to the first condition characteristic Z1 allows continuation of operation. Exemplarily it is illustrated in FIG. 10 that the second condition characteristic Z2 comprises deviations compared with reference characteristic R that indicate a non-tolerable change, e.g. contamination of the distal end 25 of optical fiber cable 23.

Due to a contamination, the reflectance at the first light wavelength λ1 and the second light wavelength λ2 decreases remarkably in the embodiment where the emission light Le has the first maximum M1 or the second maximum M2. For example, one or multiple thresholds of the transfer characteristic U can be defined that define a maximum allowed deviation from the reference characteristic R. For different light wavelengths λ different thresholds T1, T2 can be defined respectively, as apparent in FIG. 10 by way of example. For example, a first threshold T1 can be assigned to the first light wavelength λ1 and a second threshold T2 can be assigned to the second light wavelength λ2. In addition or as an alternative, it is also possible to define a threshold progress depending on the light wavelength instead of a constant threshold for each light wavelength.

The invention refers to a method V and a system 10 that is configured to determine a condition of an optical fiber cable 23 of an electrosurgical instrument 12 with regard to its transfer characteristic U for light. For this purpose system 10 can comprise an evaluation unit 27 having a light source 34 and a light analysis unit 28, which can be optically coupled with the optical fiber cable 23, particularly by means of a fiber bundle 38 and a coupling unit 26 according to another independent aspect according to the invention. The emission light Le emitted by the light source is coupled into the optical fiber cable 23 at the distal end of which it is reflected and transmitted back to the light analysis unit 28 and received there as receipt light Lr. The receipt light Lr is correlated with the emission light Le and therefrom a transfer characteristic U can be determined. If this is carried out prior to the first use of the electrosurgical instrument 12 and subsequently at least once changes in the transfer characteristic U can be determined thereby and therefrom the condition of the optical fiber cable 23 can be derived in turn.

The optical fiber cable 23 of instrument 12 is particularly directly or indirectly via an additional optical fiber cable 21 of supply apparatus 11 to the coupling unit 26 and optically coupled with a fiber bundle 35 that comprises at least one optical fiber 36 and at least one optical fiber 37. The at least one first optical fiber 36 connects the coupling unit 26 with the light source 34 and the at least one second optical fiber 37 connects the coupling unit 26 with the light analysis unit 28.

### LIST OF REFERENCE SIGNS

10 system
11 supply apparatus
12 electrosurgical instrument
13 electrode
14 electrical conductor
15 connection element
16 cable
17 handle piece
18 operating element
19 energy source
20 apparatus connection
21 optical fiber cable of the supply apparatus
23 optical fiber cable of the electrosurgical instrument
24 proximal end of the optical fiber cable of the electrosurgical instrument
25 distal end of the optical fiber cable of the electrosurgical instrument
26 coupling unit
26a first connection of coupling unit
26b second connection of coupling unit
26c third connection of coupling unit
27 evaluation unit
28 light analysis unit
29 spectrometer
30 slit 34 light source 35 fiber bundle 36 first optical fiber 37 second optical fiber 38 arrangement for mode mixing
λ light wavelength
λ1 first wavelength
λ2 second wavelength
d1 first core diameter
d2 second core diameter
d3 third core diameter
L light from tissue
Le emission light
Lr receipt light
M1 first maximum
M2 second maximum
R reference characteristic
s distance
T1 first threshold
T2 second threshold
U transfer characteristic
V method
V1 first method step
V2 second method step
V3 third method step
V4 fourth method step
V5 fifth method step
V6 sixth method step
V7 seventh method step
V8 eighth method step
V9 ninth method step
V10 tenth method step
Z condition characteristic
Z1 first condition characteristic
Z2 second condition characteristic

1. A method for determination of a condition of an optical fiber cable (23) of an electrosurgical instrument (12) comprising at least one electrode (13), wherein the electrosurgical instrument (12) is configured to be electrically connected with an energy source (19) of a supply apparatus (11) for supply of the at least one electrode (13) with electrical energy and the optical fiber cable (23) is configured to be optically connected with an evaluation unit (27), the method comprising:
connecting the electrosurgical instrument (12) in an initial condition with the energy source (19) and the evaluation unit (27);
determining a transfer characteristic (U) as a reference characteristic (R), wherein determining the transfer characteristic (U) comprises:
emitting emission light (Le) of a light source (34) of the evaluation unit (27) and coupling the emission light (Le) at least partly in a proximal end (24) of the optical fiber cable (23);
receiving receipt light (Lr) in the evaluation unit (27) that is created by transmission of the emission light (Le) via the proximal end (24) to a distal end (25) of the optical fiber cable (23), reflection at least in part at the distal end (25) of the optical fiber cable (23) and transmission from the distal end (25) via the proximal end (24) of the optical fiber cable (23) to the evaluation unit (27) coupled thereto; and
determining the transfer characteristic (U) based on the emission light (Le) and the receipt light (Lr);
activating the electrosurgical instrument (12), wherein the at least one electrode (13) is supplied with electrical energy of the energy source (19);
deactivating the electrosurgical instrument (12) by switching off the supply of electrical energy to the at least one electrode (13);
determining again a transfer characteristic (U) as a condition characteristic (Z), wherein determining the transfer characteristic (U) comprises:
emitting emission light (Le) of the light source (34) of the evaluation unit (27) and coupling the emission light (Le) at least partly in the proximal end (24) of the optical fiber cable (23);
receiving receipt light (Lr) in the evaluation unit (27) that is created by transmission of the emission light (Le) via the proximal end (24) to the distal end (25) of the optical fiber cable (23), reflection at least in part at the distal end (25) of the optical fiber cable (23) and transmission from the distal end (25) via the proximal end (24) of the optical fiber cable (23) to the evaluation unit (27) coupled thereto; and
determining the transfer characteristic (U) based on the emission light (Le) and the receipt light (Lr); and
comparing the reference characteristic (R) with the condition characteristic (Z) for determination of a current condition of the optical fiber cable (23).

2. The method according to claim 1, further comprising determining the condition characteristic (Z) and the current condition of the optical fiber cable (23) after each activation and deactivation of the electrosurgical instrument (12).

3. The method according to claim 1, further comprising receiving at least partly light that is created by spark or light arc formation at the at least one electrode (13) at the distal end (25) of the optical fiber cable (23) and transmitting the light to the evaluation unit (27).

4. The method according to claim 1, wherein the light source (34) is only activated for emission of emission light (Le), if no electrical energy is transmitted to the at least one electrode (13).

5. The method according to claim 1, wherein the emission light (Le) of the light source (34) comprises light wavelengths in a range lower than 380 nm.

6. The method according to claim 1, wherein the emission light (Le) of the light source (34) comprises multiple maxima (M1, M2) at different light wavelengths (λ).

7. The method according to claim 1, wherein the evaluation unit (27) comprises a light analysis unit (28), wherein the light source (34) is connected to a first connection (26a) of a coupling unit (26), the light analysis unit (28) is connected to a second connection (26b) of the coupling unit (26) and the optical fiber cable (23) is connected to a third connection (26c) of the coupling unit (26).

8. The method according to claim 7, wherein the first connection (26a) and the second connection (26b) of the coupling unit (26) are optically separated from one another and optically separately connected to the third connection (26c) of the coupling unit (26).

9. The method according to claim 8, further comprising determining that the optical fiber cable (23) of the electrosurgical instrument (12) is connected to the third connection (26) of the coupling unit (26) by coupling emission light (Le) in the first connection (26a) of the coupling unit (26) and by receiving receipt light (Lr) in the evaluation unit (27).

10. A system (10), comprising:
an energy source (19) of a supply apparatus;
an evaluation unit (27); and
an electrosurgical instrument;
wherein the evaluation unit (27) comprises a light source (34) and a light analysis unit (28);
wherein the electrosurgical instrument (12) comprises at least one electrode (13) that is electrically connected with the energy source (19);
wherein the electrosurgical instrument (12) comprises an optical fiber cable (23) that is optically coupled with the evaluation unit (27) by means of a coupling unit (26);
wherein the light source (34) is connected to a first connection (26a) of the coupling unit (26), the light analysis unit (28) is connected to a second connection (26b) of the coupling unit (26) and the optical fiber cable (23) is connected to a third connection (26c) of the coupling unit; and
wherein the first connection (26a) and the second connection (26b) of the coupling unit (26) are optically separated from one another and are separately optically connected with a third connection (26c) of the coupling unit (26).

11. The system according to claim 10, wherein the coupling unit (26) comprises a fiber bundle (35) having at least one first optical fiber (36) and at least one second optical fiber (37), wherein the at least one first optical fiber (36) optically connects the first connection (26a) of the coupling unit (26) with the third connection (26c) of the coupling unit (26) and the at least one second optical fiber (37) optically connects the second connection (26b) of the coupling unit (26) with the third connection (26c) of the coupling unit (26).

12. The system according to claim 11, wherein the at least one first optical fiber (36) and the at least one second optical fiber (37) have a smaller core cross-sectional area than the optical fiber cable (23).

13. The system according to claim 11, wherein a core cross-sectional area of the at least one second optical fiber (37) is at most 25% of a core cross-sectional area of the optical fiber cable (23).

14. The system according to claim 11, wherein a core cross-sectional area of the at least one first optical fiber (36) is smaller than a core cross-sectional area of the at least one second optical fiber (37).

15. The system according to claim 10, comprising an arrangement for mode mixing (38) between the evaluation unit (27) and the coupling unit (26).
